(19) United States
(12) Patent Application Publication
(10) Pub. No.: US 2018/0055920 A1
(43) Pub. Date: Mar. 1, 2018

(54) VACCINE, THERAPEUTIC COMPOSITION AND METHODS FOR TREATING OR INHIBITING CANCER

(71) Applicant: UNIVERSITY OF IOWA RESEARCH FOUNDATION, Iowa City, IA (US)

(72) Inventor: Gail Bishop, Iowa City, IA (US)

(73) Assignee: UNIVERSITY OF IOWA RESEARCH FOUNDATION, Iowa City, IA (US)

(21) Appl. No.: 15/557,553

(22) PCT Filed: Mar. 11, 2016
(86) PCT No.: PCT/US16/21955

§ 371 Date: Sep. 12, 2017

Related U.S. Application Data
(60) Provisional application No. 62/132,316, filed on Mar. 12, 2015.

(51) Int. Cl. A61K 39/00; A61K 45/06

(52) U.S. Cl. CPC A61K 39/0011; G01N 33/574; C07K 2317/73; A61K 45/06

(57) ABSTRACT

The present invention provides a method of preparing an anti-cancer composition comprising activated immune cells and a pharmaceutically acceptable excipient. The present invention further provides a method of contacting immune cells obtained from an animal with an optimal combination of activating agents and with an immunogenic material to form the activated immune cells.

### RELATED APPLICATION

This application claims priority to U.S. Provisional Patent Application No. 62/132,316, filed Mar. 12, 2015, the entirety of which is incorporated herein by reference.

### GOVERNMENT FUNDING

This invention was made with government support under BX001702 awarded by Office of Veterans Affairs. The government has certain rights in the invention.

### BACKGROUND OF THE INVENTION

Immunotherapy is a treatment that uses components of a person's immune system to fight disease. To target cancer, the patient's immune system is stimulated to work harder or to better target specific cancerous cells.

Cancer vaccines are made up of cancer cells, parts of cells, or pure antigens (immunogens). In some cancer vaccines, a patient's own immune cells are removed and exposed to these substances in a lab. Once the vaccine is ready, it is injected into the body to increase the immune response against cancer cells. This immune stimulation causes the immune system to attack tumor cells.

Currently, the only FDA-approved cellular vaccine to treat cancer is PROVENGE® (sipuleucel-T), which is used to treat advanced prostate cancer by separating immature dendritic cells (DC; a type of antigen-presenting cell) from the body, activating the cells with recombinant antigen for prostate cancer, and infusing the now mature cells back into the patient's body. Once inside the body, these cells then activate the body's resting T cells. These T cells then recognize and attack prostate cancer cells. Although this approach looked very promising in preclinical studies in mice, it has proven disappointing in humans. Feasibility issues that are addressed by this invention may be limiting the efficacy of dendritic cell vaccines in humans. These include the difficulty in obtaining an optimal number of such cells, because they are present in small numbers in the peripheral blood, and the challenges of maturing the cells in culture, which is time-consuming and laborious.

Despite advances in cancer research, there are still no adequate treatments for many cancers. Accordingly, new compositions and methods to treat cancer are needed.

### SUMMARY OF THE INVENTION

Current techniques for immunotherapy of cancer rely on use of a patient's dendritic cells for a cellular vaccine. This has caused problems as it is challenging to obtain sufficient numbers of dendritic cells for effectiveness as these cells are not common in the peripheral blood and mature DC cannot be replicated outside the body using current techniques.

The present invention provides a method of preparing an anti-cancer composition comprising activated immune cells and a pharmaceutically acceptable excipient, the method comprising contacting immune cells obtained from an animal with an optimal combination of activating agents and with an immunogenic material obtained from the tumor to form the activated immune cells. The present invention provides in certain embodiments a method of preparing an anti-cancer composition comprising activated cells and a pharmaceutically acceptable excipient, the method comprising: obtaining immune cells from an animal, obtaining immunogenic material from the animal's tumor, incubating the cells with an activating agent, and incubating the cells with the immunogenic material to generate the activated cells.

The present invention provides in certain embodiments pharmaceutical composition comprising immune cells obtained from an animal wherein the cells have been stimulated ex vivo with an activating agent and have been stimulated ex vivo with an immunogenic material obtained from the animal's tumor for the therapeutic treatment of cancer.

The present invention provides in certain embodiments a method of treating cancer in an animal in need thereof comprising administering to the patient the composition described herein.

The present invention provides in certain embodiments a method comprising administering a dendritic cell-based vaccine followed by administering the composition described herein. As used herein, the term “dendritic cell-based vaccine” is a vaccine that is produced by removing dendritic cells from a patient, maturing the DCs in vitro, exposing the DCs to a stimulating agent, and then administering the stimulated DCs back to the patient. In certain embodiments, the method further comprising administering repeat dosages of the composition described herein.

The present invention provides in certain embodiments a method of eliciting an immune response in an animal in need thereof, comprising administering to the animal the composition described herein.

The present invention provides in certain embodiments a use of the anti-cancer composition described herein for treating cancer.

The present invention provides in certain embodiments a use of a therapeutic composition described herein to prepare a medicament for treating cancer in an animal, such as a human.

### BRIEF DESCRIPTION OF THE FIGURES

FIG. 1. Bvac Experimental Design: Splenic B cells are harvested, variably stimulated for 24 hours and pulsed with a source of tumor-derived antigen (OVA, or 1-3 melanoma peptides or tumor lysate) for 22 hours. Bvac is given on day 0. On day 14, a Bvac booster is given. Each Bvac contains 2*10⁵ stimulated B cells. On day 40, mice are challenged with 5*10⁴ melanoma cells.

FIG. 2: Trafficking of activated B cells after intravenous injection. Splenic B cells were isolated from naïve CD45.1+C57BL/6 mice and activated through CD40 and TLR4 for 24 hours. 1×10⁶ activated B cells were injected i.v. into naïve CD45.2+C57BL/6 recipients. At the indicated times, spleens and lymph nodes were collected from euthanized mice. CD45.1+B cells in secondary lymphoid organs were quantified by surface staining for B220 and CD45.1 and were analyzed by flow cytometry. The bar graph represents six individual mice.

FIGS. 3A-C: FIG. 3A. Use of Bvacs against two clones of B16 murine melanoma. Purified B cells were activated through the B cell antigen receptor (BCR) and the innate immune receptor Toll-like receptor 7 (TLR7) for 24 hours in culture (Vanden Bush, T., Buchta, C. M., Claudio, J., Bishop, G. A. (2009) Cutting Edge: Importance of IL-6 and cooperation between innate and adaptive immune receptors in cellular vaccination with B lymphocytes. J Immunol 183, 4833-4837). Unstimulated B cells were used as a negative control. Cells were pulsed with either tyrosinase or SIINFEKL peptide and 2×10⁵ cells were injected i.v. into naïve mice. On day 40 after vaccination, mice that received tyrosinase-pulsed Bvacs were injected with 5×10⁴ B16F1 cells subcutaneously in the right flank. Mice that received SIINFEKL-pulsed Bvacs were injected with 5×10⁴ B16-OVA cells. Tumor growth was measured with calipers every other day. The graph represents tumors on all mice measured at day 18 after tumor injection.

FIG. 3B. Bvac induction of CD8+ cytolytic T cell activation. Bvac was performed as in FIG. 2A, using B16-Ova tumor cells. On day 4 post-tumor cell injection, recall responses of splenic CD8 T cells were examined by intracellular staining for the cytokine interferon gamma (IFNγ). Plots are representative of five mice.

FIG. 3C. Booster vaccines increase CD8 T cell response. Bvac was performed as in FIGS. 3A-3B, with some groups receiving booster injections of the same B cell numbers on day 7 or day 14. T cell responses were measured as in FIG. 3B.

FIG. 4: Optimizing Bvac stimulation for use against murine melanoma. Splenic B cells were purified as in prior Figures, and activated through the indicated receptors for 24 hours in culture. Cells were pulsed with antigenic peptide (SIINFEKL) and 2×10⁵ cells were injected i.v. into naïve mice. On day 14 after vaccination, all mice received a booster Bvac stimulated through the same receptors as the Bvac given at day 0. On day 40 after vaccination, mice were injected with 5×10⁴ B16-OVA cells subcutaneously in the right flank. Tumor growth was measured with calipers every other day for a period of 60 days. Mice were euthanized when tumor growth measured 150 mm². Each graph represents combined data from two separate experiments with five mice per group.

FIGS. 5A-5C. FIG. 5A: Refinement of adjuvant to increase clinical relevance. FIG. 5A, the best adjuvant combination was used with three endogenous peptides or tumor lysate.

In FIG. 5B, the best adjuvants in FIG. 4 were selected, and used in a Bvac with the tumor specific peptide TRP2 or a lysate prepared from the melanoma cells, to protect the mice against the B16F1 tumor (which does not express the exogenous OVA peptide SIINFEKL).

FIG. 5C. Tumor sizes of experiment plotted in FIG. 5B. FIG. 6: PLGA blank Nanoparticles.

FIG. 6. PLGA blank Nanoparticles.

FIG. 7: Tumor cell lysate is an effective source of Bvac stimulating antigen. Bvac was prepared and delivered as in FIGS. 2-4, but the B16F1 parent tumor was used, with Bvac stimuli of CD40/TLR4/TLR7+a combination of 3 purified melanoma peptides (TRP-2, Mage-A5, Mage-Ax) or B16 lysate, prepared as in Gross, BP, Wongrakpanich, A, Francis, M B, Salem, A K & Norian, L A. A therapeutic microparticle-based tumor lysate vaccine reduces spontaneous metastases in murine breast cancer. AAPS J. 16:1194-1203, 2014.

FIG. 8: Upregulation of Bvac surface molecules. Bvac were incubated for 24 h in medium only (left panel of each indicated pair of panels) or a mixture of tumor cell lysate+stimuli through CD40, TLR4, & TLR7, as in FIG. 7 (righthand panel of each pair above). Cells were stained for expression of MHC class I, CD80 and PDL-1, as indicated under each pair of panels. Similar results were seen when staining for MHC class II, LFA-1 and ICAM-1 (not shown).

### DETAILED DESCRIPTION OF THE INVENTION

B Lymphocytes (B cells) are a specialized type of white blood cell that can take up antigens either through specific recognition receptors, or through nonspecific means (endocytosis). Once the B cell connects to the antigen, the cell consumes and processes it. The processed parts of the antigen are placed in the binding pocket of a self-recognition molecule called a Major Histocompatibility Complex (MHC) molecule. If the B cell also receives nonspecific “innate” immune signals from receptors for these signals, it becomes activated and expresses molecules on its surface that attracts T cells. The T cell expresses receptors that recognize the antigen-MHC complex on the B cells, and also the costimulatory molecules expressed by the activated B cell. Some of these T cells are specialized to stimulate the B cell to produce antibodies. Other T cells (cytotoxic T lymphocytes, or CTL) are activated to produce granules containing substances that can kill target cells—such as tumor cells—recognized by the activated T cell.

It is comparatively much easier to isolate B cells from blood than dendritic cells; the latter are rare in blood, while B cells are abundant. Additionally, dendritic cells are end-stage cells that divide little if at all after removal, while B cells can be easily induced to multiply in culture.

### Use of B Cells as APCs in Cellular Vaccines

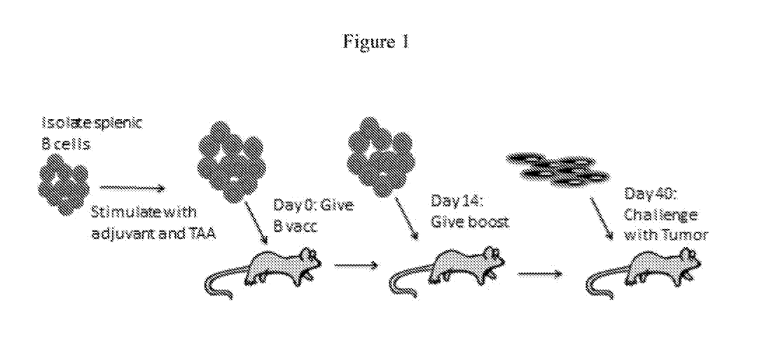

Cellular vaccines present a unique strategy to fight tumors. The current paradigm for cellular vaccines using dendritic cells has failed to meet expectations in clinical trials. Limitations of DCvacs include low number of DCs in peripheral blood, difficulty of isolation, and difficulty of ex vivo expansion. B cell vaccines are advantageous due to ease of B cell isolation and expansion. B cells also function as effective antigen presenting cells when optimally stimulated. It has been previously shown that Bvac is effective in protecting mice from a specific pathogen and in promoting a specific CD8 T cell response. (FIG. 1)

Although most work on the innate immune receptors called toll-like receptors (TLRs) focuses specifically on myeloid cells, TLRs are important signaling receptors in B lymphocytes. TLR stimulation of B cells enhances B cell effector function and results in cellular activation.

Current research aims to develop new immunotherapeutics for cancer through cellular vaccines. The majority of groups pursuing these approaches are utilizing dendritic cells (DCs) as the antigen-presenting cell (APC) in cellular vaccines due to the breadth of knowledge available on DC-mediated antigen presentation. However, B lymphocytes express MHC class I and II molecules and effectively present antigen to T cells. The ability to present antigen to T cells, the relative ease of isolation from peripheral blood, and the potential for in vitro activation and expansion make activated B lymphocytes an attractive alternative to DCs in human immunotherapy. Many studies in both DCs and B cells utilize TLR agonists to activate and differentiate the APCs for use in cellular vaccines. Signaling through TLRs synergizes with signaling through other receptors such as CD40 and the B cell antigen receptor (BCR), resulting in greater cellular activation than stimulation through any single receptor.

The present inventors compared DCvacs to Bvacs in their ability to induce protection in a model of bacterial infection. DCvacs outperformed Bvacs in terms of in vitro IFNγ production by memory CD8⁺ T cells in a peptide recall response assay. However, bacterial clearance is a more physiologically relevant measure of vaccine efficacy, and the immune response elicited by Bvac-vaccinated mice is equivalent to that elicited by DCvac-vaccinated mice. This adds support to literature demonstrating that in vitro responses, particularly induced expression of IFNγ, are not always a reliable indicator of how cells will perform in vivo. These data show that activated B cells can act as efficient cellular vaccines and promote memory CD8⁺ T cell responses equivalent to those elicited by DCvacs.

The optimal method for activating B cells as APCs in cellular vaccines may differ depending on the model used. Activating B cells through BCR+TLR7 was most efficacious in a model of protection against Listeria monocytogenes, but stimulation through CD40+TLR4 or CD40+TLR7 trended towards being more effective in a model of protection against murine melanoma. Thus, in certain embodiments, a triple stimulus of CD40+TLR7+TLR4 is used. After determining the optimal stimuli to activate B cells against infectious disease, the Bvac system was used against cancer in a model of murine melanoma. BCR+TLR7-stimulated Bvacs were efficient in delaying tumor growth in two models of B16 melanoma: parent B16F1 cells, and B16 cells that express chicken ovalbumin. In addition to BCR+TLR7-stimulated Bvacs, an additional Bvac stimulus was identified that provides effective protection against tumor development and growth: stimulation through CD40 and TLR4. Both BCR+TLR7- and CD40+TLR4-stimulated Bvacs significantly delayed tumor onset and progression while 30% of the mice in the BCR+TLR7 Bvac group and 50% of the CD40+TLR4 Bvac group never developed a palpable tumor, even at 60 days after tumor injection. Recent experiments indicate that the best result is obtained with a triple adjuvant stimulus of CD40+TLR7+TLR4.

Additionally, IL-6 production was identified as a key component in efficacious cellular vaccines against an infectious pathogen. Bvacs created from IL-6 KO B cells were not as effective as WT B cells in eliciting CD8⁺ T cell responses, and blocking IL-6 signaling in culture resulted in decreased CD8⁺ T cell proliferation. BCR+TLR7-stimulated B cells produced the most IL-6 in culture, and it was hypothesized that this increased cytokine production directly impacts the superior in vivo responses seen with these Bvacs in a model of infectious disease. If additional biomarkers of efficacious cellular vaccines are discovered, the need for cellular vaccines may be eliminated altogether. An alternate strategy would utilize a synthetic nanoparticle system containing the necessary adjuvants, antigen, cytokines, and/or costimulatory molecules or signals.

Much of the current work with DCvacs focuses on combining cellular vaccines with additional therapies against cancer. Future directions may utilize Bvacs in combination with chemotherapeutic drugs and/or blocking monoclonal antibodies against targets such as the T cell-inhibiting surface molecules PD-1, CTLA-4, and Tim-3. A recent report found that peptide vaccines against melanoma induce antigen-specific CD8⁺ T cell responses; however the vaccine-induced T cells upregulated both PD-1 and Tim-3. Blocking both PD-1 and Tim-3 in vitro enhanced CD8⁺ T cell expansion and cytokine production. Use of additional therapies may boost Bvac effectiveness in promoting immune responses against cancer.

### Methods of Making Cancer Vaccines

The present invention provides a method of preparing an anti-cancer composition comprising contacting immune cells obtained from an animal with an activating agent and with an immunogenic material obtained from the animal's tumor to form an anti-cancer composition, wherein the activated immune cells are a population of cells that are at least 90% B-lymphocytes.

The present invention provides in certain embodiments a method of preparing an anti-cancer composition comprising: obtaining immune cells from an animal, obtaining immunogenic material from the animal, contacting the immune cells with a preparation of activating agents, and contacting the immune cells with the immunogenic material, wherein the activated immune cells are a population of cells that are at least 90% B-lymphocytes.

In certain embodiments, the present invention further comprises purifying the activated B cells away from the activating agent and the immunogenic material.

In certain embodiments, the immune cells are a population of cells that are at least 90% B-lymphocytes.

In certain embodiments, the immune cells are at least 91%, 92%, 93%, 94%, 95%, 96%, 97%, 98%, 99% or 100% B-lymphocytes.

In certain embodiments, the immune cells have been expanded in vitro prior to administration into the animal. As used herein, the term “expanded” means the production of daughter cells arising originally from a single cell. In a clonal expansion of lymphocytes, the progeny share the same antigen specificity.

In certain embodiments, the immune B cells from the animal have been stimulated ex vivo with an activating agent and have been stimulated ex vivo with an immunogenic material obtained from the animal's tumor.

In certain embodiments, the composition is substantially devoid of activating agent and/or immunogenic material.

In certain embodiments, the immune cells are B-lymphocytes and/or antigen presenting cells (APCs).

In certain embodiments, the immune cells are stimulated ex vivo.

In certain embodiments, the immune cells are stimulated for about 1-72 hours. In certain embodiments, the immune cells are stimulated for about 12-60 hours. In certain embodiments, the immune cells are stimulated for about 24-48 hours.

In certain embodiments, the immune cells are obtained from peripheral blood from the animal.

In certain embodiments, the therapeutic composition further comprises an anti-cancer therapeutic.

In certain embodiments, the composition further comprises a physiologically-acceptable, non-toxic vehicle or pharmaceutically acceptable excipient.

In certain embodiments, the composition is substantially devoid of activating agent and/or immunogenic material. As used herein, the term “substantially devoid” means that the composition contains less than 10% (i.e., less than 9%, 8%, 7%, 6%, 5%, 4%, 3%, 2%, 1%, 0.5%, 0.1%) activating agent and/or immunogenic material.

In certain embodiments, the composition further comprises an anti-cancer therapeutic.

### Activating Agents

In certain embodiments, the activating agent comprises a CD40 agonist, BCR agonist, toll-like receptor 7 (TLR7) agonist, a TLR4 agonist, AS04, alum, TLR3 agonist and/or a TLR9 agonist. In certain embodiments, the activating agent comprises various combinations of the agents listed above. In certain embodiments, the activating agent comprises a CD40 agonist, TLR7 agonist, and a TLR4 agonist. As used herein, the term “agonist” is a stimulating agent.

### Immunogenic Material

In certain embodiments, the immunogenic material comprises tumor cells or a tumor cell lysate from the animal.

In certain embodiments, the immunogenic material comprises purified proteins or peptides from the tumor cells.

In certain embodiments, the tumor cells are solid tumor cells or are hematopoietic cancer cells.

In certain embodiments, the tumor cells are lysed. Tumor lysates are made by extracting a sample of the tumor to be treated from the subject. The tumor cells are then lysed. Methods of making effective tumor lysates include, but are not limited to, freeze thaw method, sonication, microwave, boiling, high heat, detergent or chemical-based cell lysis, electric or current-based lysis, and other physical methods, such as extreme force.

In certain embodiments, the tumor cells are irradiated.

### Encapsulating Materials

In certain embodiments, the activating agent is encapsulated in a nanoparticle.

In certain embodiments, the immunogenic material is encapsulated in a nanoparticle.

In certain embodiments, Poly(Lactide-co-Glycolide) (PLGA) particles were fabricated. Briefly, particles were prepared using double emulsion solvent evaporation method as previously described with some modifications (Gross B P, Wongrakpanich A, Francis M B, Salem A K, Norian L A: A therapeutic microparticle-based tumor lysate vaccine reduces spontaneous metastases in murine breast cancer. The AAPS journal 16(6), 1194-1203 (2014)). Water phase 1, 125 μl of aqueous phase contains 2.5% poly(vinyl alcohol) (PVA), was sonicated into an oil phase which contains 200 mg of PLGA (Resomer® RG502H) in 2.5 ml of dichloromethane (DCM) using Sonic Dismembrator Ultrasonic processor to create primary emulsion. The secondary emulsion was created by sonicating the primary emulsion in water phase 2 which is 8 mL of 2.5% PVA in MES buffer. This emulsion was poured into 22 ml of 2.5% PVA in MES buffer. DCM was evaporated via stirring the particle suspensions in the fume hood for 2 hours. Differential centrifugation was used to achieve small size particles with narrow poly dispersity index. Large particles were separated out by centrifuge at 800 g for 5 mins. Supernatant obtained after this step contained small size particles. These particles were collected using centrifugation at 10 Kg for 30 mins, washed three times with water, and lyophilized. Blank particles are particles which made from 2.5% PVA in water phase 1. Rhodamine B loaded particles are particles which made from rhodamine B 1 mg in 2.5% PVA in oil phase.

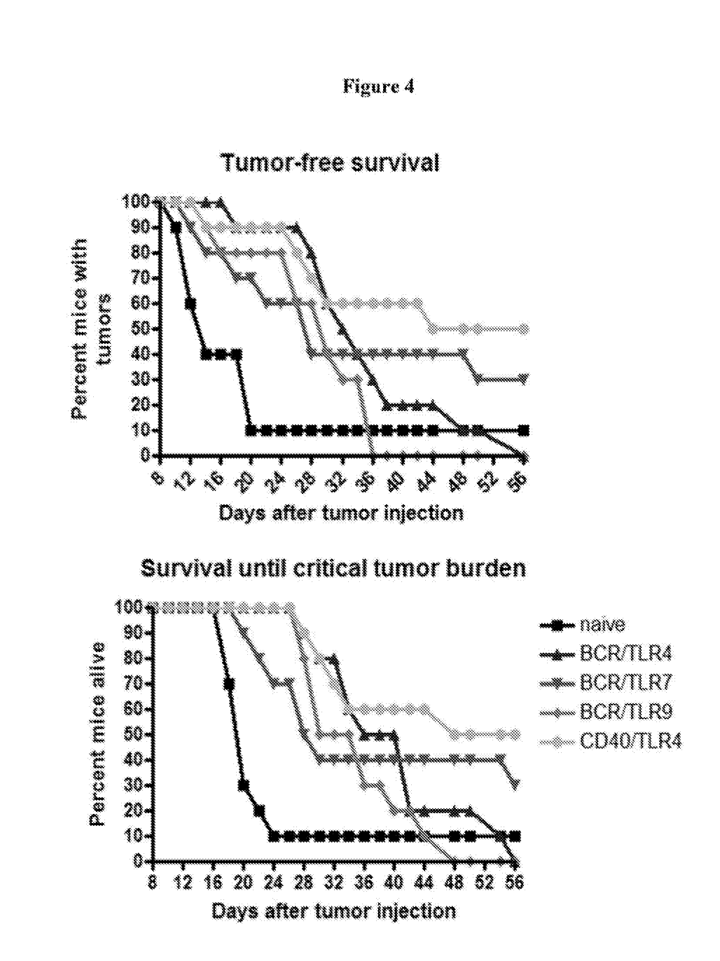

Size and zeta potential of lyophilized particles were measured using Zetasizer nano ZS. Scanning Electron Microscopy (SEM) was used to observe the particles' morphology. Weight of the particles in each batch obtained from weight of the tube with particles after lyophilization minus weight of the tube alone. The results of two batches of PLGA blank particles are provided in Table 1 below (FIG. 4).

TABLE 1

| | Blank particles (1^{st} batch) | | Blank particles (2^{nd} batch) |
|---|---|---|---|
| Z-average | 392.2 | | 354.3 |
| Zeta-potential | −23.4 | mV | −21.8 mV |
| PdI | 0.608 | | 0.165 |
| Weight | 130.9 | mg | 135.3 |

### Adjuvants

In certain embodiments, the composition further comprises an adjuvant. An “adjuvant” is a molecule or compound that stimulates the humoral and/or cellular immune response in an antigen-independent way. Substances with adjuvant properties are considered to be ‘nonspecific’ in that all immune cells of given types have receptors for them, regardless of whether they have specific antigen receptors, or what the specificity is of those antigen receptors. Adjuvants allow much smaller doses of antigen to be used and are essential to inducing a strong antibody response to soluble antigens. In certain embodiments, the therapeutic agent is administered in conjunction with one or more adjuvants (i.e., simultaneously with the therapeutic agent).

Vaccines commonly contain two components: antigen and adjuvant. The antigen is the molecular structure encoded by the pathogen or tumor against which the immune response is directed. To activate an antigen-specific immune response, the antigen must be presented in the appropriate immunostimulatory microenvironment. In certain embodiments, adjuvants establish such microenvironments by stimulating the production of immune-activating molecules such as proinflammatory cytokines. Vaccine efficacy depends on the types of antigen and adjuvant, and how they are administered. Striking the right balance among these components is important to eliciting protective immunity.

In certain embodiments, the adjuvant is a TLR ligand. In certain embodiments, the adjuvant is an agonistic monoclonal antibody (mAb) specific for CD40. In certain embodiments, the adjuvant is a non-TLR ligand that stimulates an immune response.

### Toll-Like Receptors

It has been estimated that most mammalian species have between ten and fifteen types of Toll-like receptors (TLRs). Eleven TLRs (named simply TLR1 to TLR11) have been identified in humans, and equivalent forms of many of these have been found in other mammalian species. TLRs function as a dimer. Though most TLRs appear to function as homodimers, TLR2 forms heterodimers with TLR1 or TLR6, each dimer having different ligand specificity. The function of TLRs in all organisms appears to be similar enough to use a single model of action. Each Toll-like receptor forms either a homodimer or heterodimer in the recognition of a specific or set of specific molecular determinants present on microorganisms

TLRs sense infection by recognizing pathogen associated molecular patterns and/or “danger”′ signals perceived by immune cells, and triggering immune cell activation in an antigen-nonspecific manner. Therefore TLR ligands have been developed as vaccine adjuvants. In certain embodiments a ligand to TLR1 through TLR11 may be used as an adjuvant. Antigen-presenting cells (APC) that engulf antigen may also take up TLR ligand, resulting in up-regulation of co-stimulatory molecules, secretion of inflammatory cytokines, and presentation of antigen to T cells. This is certainly the case when APCs process viral particles, which contain both TLR ligands (e.g., viral RNA, the natural ligand for TLR7) and viral proteins. Some TLRs are located on the cell surface, and thus their ligands do not need to be internalized (e.g., TLR4). In the case of TLR7, small molecule mimics of the natural viral nucleic acid agonist can be used (e.g., R848). However, in the case of cancer vaccines the antigen and TLR ligand have been administered in mixture.

### Cancers

In certain embodiments, the composition described above is used in the manufacture of a medicament for treating a disease or disorder arising from abnormal cell growth, function or behavior. In certain embodiments, the disease or disorder is cancer.

In certain embodiments, the cancer is selected from solid tumors of the colon, breast, brain, liver, ovarian, gastric, lung, and head and neck. In certain embodiments, the cancer is selected from glioblastoma, melanoma, prostate, endometrial, ovarian, breast, lung, head and neck, hepatocellular, and thyroid cancers. In certain embodiments, the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's lymphoma and leukemia. In certain embodiments, the cancer is a haematopoietic cancer.

### Formulations

Vaccine formulations will contain an effective amount of the active ingredient in a vehicle, the effective amount being readily determined by one skilled in the art. The active ingredient may typically range from about 1% to about 95% (w/w) of the composition, or even higher or lower if appropriate. The quantity to be administered depends upon factors such as the age, weight and physical condition of the animal or the human subject considered for vaccination. Effective dosages can be readily established by one of ordinary skill in the art through routine trials establishing dose response curves.

In certain embodiments, anti-tumor composition formulations contain an effective amount of the stimulated immune cells (the “active ingredient”) in a vehicle, the effective amount being readily determined by one skilled in the art. The active ingredient may typically range from about 1% to about 95% (w/w) of the composition, or even higher or lower if appropriate. The quantity to be administered depends upon factors such as the age, weight and physical condition of the animal or the human subject considered for vaccination. Effective dosages can be readily established by one of ordinary skill in the art through routine trials establishing dose response curves. Multiple doses may be administered as is required.

In certain embodiments, to prepare an anti-tumor composition, the stimulated immune cells are prepared as described above. The amount of stimulated immune cells is then be adjusted to an appropriate concentration, optionally combined with a suitable adjuvant, and packaged for use.

The pharmaceutical dosage forms suitable for injection or infusion can include sterile aqueous solutions or dispersions comprising the active ingredient, which are adapted for the extemporaneous preparation of sterile injectable or infusible solutions or dispersions. In all cases, the ultimate dosage form should be sterile, fluid and stable under the conditions of manufacture and storage. The liquid carrier or vehicle can be a solvent or liquid dispersion medium comprising, for example, water. In many cases, it will be preferable to include isotonic agents, for example, sugars, buffers or sodium chloride. Sterile injectable solutions are prepared by incorporating the active compound in the required amount in the appropriate solvent with the various other ingredients.

The amount of the active ingredient required for use in treatment will vary not only with the particular vaccine preparation but also with the nature of the condition being treated and the age and condition of the patient, and will be ultimately at the discretion of the attendant physician or clinician.

### Modes of Administration

In certain embodiments, the present invention provides in certain embodiments a method of treating cancer in an animal in need thereof comprising, administering to the patient the composition as described herein.

The present invention provides in certain embodiments a method of eliciting an immune response in an animal in need thereof, comprising administering to the animal the composition as described herein.

In certain embodiments, the administration is by means of injection.

In certain embodiments, the composition is administered intravenously or intra-tumorally.

In certain embodiments, the anti-tumor composition is administered at more than one time point.

In certain embodiments, the anti-tumor composition is administered at two to five time points (i.e., 2, 3, 4, or 5 time points).

In certain embodiments, the animal is a mammal. In certain embodiments, the mammal is a human.

The present invention provides in certain embodiments a use of the anti-cancer composition as described herein for treating cancer.

The present invention provides in certain embodiments a use of a therapeutic composition as described herein to prepare a medicament for treating cancer in an animal.

The vaccines and compositions of the invention may be formulated as pharmaceutical compositions and administered to a mammalian host, such as a human patient, in a variety of forms adapted to the chosen route of administration, i.e., orally, by intravenous, routes. The active compound may also be administered intravenously or intratumorally by infusion or injection. None of the material above is applicable to cellular vaccines.

In certain embodiments, the present invention provides a method comprising administering a dendritic cell-based vaccine followed by administering the composition as described herein to an animal in need thereof.

“Effective amount” or “therapeutically effective amount” of a compound refers to a nontoxic but sufficient amount of the compound to provide the desired therapeutic or prophylactic effect to most patients or individuals. In the context of treating cancer, a nontoxic amount does not necessarily mean that a toxic agent is not used, but rather means the administration of a tolerable and sufficient amount to provide the desired therapeutic or prophylactic effect to a patient or individual. The effective amount of a pharmacologically active compound may vary depending on the route of administration, as well as the age, weight, and sex of the individual to which the drug or pharmacologically active agent is administered Those of skill in the art given the benefit of the present disclosure can easily determine appropriate effective amounts by taking into account metabolism, bioavailability, and other factors that affect plasma levels of a compound following administration within the unit dose ranges disclosed further herein for different routes of administration.

“Treatment” or “treating” refers to any manner in which the symptoms of a condition, disorder or disease are ameliorated or otherwise beneficially altered. In the context of treating the cancers disclosed herein, the cancer can be onset, relapsed or refractory. Full eradication of the condition, disorder or disease is not required. Amelioration of symptoms of a particular disorder refers to any lessening of symptoms, whether permanent or temporary, that can be attributed to or associated with administration of a therapeutic composition of the present invention or the corresponding methods and combination therapies. Treatment also encompasses pharmaceutical use of the compositions in accordance with the methods disclosed herein.

### Example 1

### Cooperation Between Toll-Like Receptors and Other Signals to Activate B Lymphocytes for Use in Cellular Vaccines

The ability to present Ag to naïve T cells, the relative ease of isolation from peripheral blood, and the potential for in vitro activation and expansion make B lymphocytes an attractive alternative to Dendritic cells (DCs) in human immunotherapy. Treating APCs with specific activation stimuli and Ag ex vivo followed by reintroduction into the body has been proposed as a possible immunotherapy to combat cancer and infectious diseases for which conventional vaccines have proven ineffective. Activation stimuli promote the expression of costimulatory molecules such as CD80 and CD86 on APCs and enhance production of the cytokines necessary for optimal T cell activation (Freeman, G. J., et al., Cloning of B7-2: a CTLA-4 counter-receptor that costimulates human T cell proliferation. Science, 1993. 262(5135): p. 909-11; Boussiotis, V. A., et al., B7 but not intercellular adhesion molecule-1 costimulation prevents the induction of human alloantigen-specific tolerance. J Exp Med, 1993. 178(5): p. 1753-63; Freeman, G. J., et al., Structure, expression, and T cell costimulatory activity of the murine homologue of the human B lymphocyte activation antigen B7. J Exp Med, 1991. 174(3): p. 625-31).

Previous reports demonstrate that antigen-presenting B lymphocytes stimulated through the TNFR superfamily member CD40 upregulate the expression of CD86 and proinflammatory cytokines. Additionally, activated and peptide-pulsed B cells induce CD8⁺ T cell responsiveness (Lapointe, R., et al., CD40-stimulated B lymphocytes pulsed with tumor antigens are effective antigen presenting cells that can generate specific T cells. Cancer Res, 2003. 63(11): p. 2836-43; Schultze, J. L., et al., CD40-activated human B cells: an alternative source of highly efficient antigen presenting cells to generate autologous antigen-specific T cells for adoptive immunotherapy. J Clin Invest, 1997. 100(11): p. 2757-65; Constant, S. L., B lymphocytes as antigen presenting cells for CD4+ T cell priming in vivo. J Immunol, 1999. 162(10): p. 5695-703; Schultze, J. L., S. Grabbe, and M. S. von Bergwelt-Baildon, DCs and CD40-activated B cells: current and future avenues to cellular cancer immunotherapy. Trends Immunol, 2004. 25(12): p. 659-64). These studies offer an important insight into the potential use of B cells in immunotherapy. However, it is generally believed that B cells are much less effective APCs than DC, and as a result, most groups have ignored their potential for use in cellular vaccines.

In addition to CD40, B cells can be activated through engagement of the BCR and innate immune receptors such as TLRs. The stimulation of B cells through these receptors can induce the expression of costimulatory molecules and the production of proinflammatory cytokines such as IL-6 and TNF-α (Vanden Bush, T. J. and G. A. Bishop, TLR7 and CD40 cooperate in IL-6 production via enhanced JNK and AP-1 activation. Eur J Immunol, 2008. 38(2): p. 400-9; Bishop, G. A., et al., The immune response modifier resiquimod mimics CD40-induced B cell activation. Cell Immunol, 2001. 208(1): p. 9-17; Baccam, M., et al., CD40-mediated transcriptional regulation of the IL-6 gene in B lymphocytes: involvement of NF-kappa B, AP-1, and C/EBP. J Immunol, 2003. 170(6): p. 3099-108; Bishop, G. A., et al., Molecular mechanisms of B lymphocyte activation by the immune response modifier R-848. J Immunol, 2000. 165(10): p. 5552-7). Although BCR stimulation alone can in some circumstances induce B cell anergy, stimulation through the BCR together with any of the multiple TLRs expressed by B cells significantly enhances B cell effector functions, including cytokine production, Ab production, and surface molecule upregulation (Bishop, G. A., et al., The immune response modifier resiquimod mimics CD40-induced B cell activation. Cell Immunol, 2001. 208(1): p. 9-17; Bishop, G. A., et al., Molecular mechanisms of B lymphocyte activation by the immune response modifier R-848. J Immunol, 2000. 165(10): p. 5552-7).

The use of TLR agonists as vaccine adjuvants is currently being tested (Vasilakos, J. P., et al., Adjuvant activities of immune response modifier R-848: comparison with CpG ODN. Cell Immunol, 2000. 204(1): p. 64-74; Weeratna, R. D., et al., TLR agonists as vaccine adjuvants: comparison of CpG ODN and Resiquimod (R-848). Vaccine, 2005. 23(45): p. 5263-70). Initial studies focused upon agonists of the endosomal TLRs 7, 8 and 9. B lymphocytes readily respond to the TLR7 agonist R848. Stimulation through TLR7 enhances B cell function and strongly synergizes with BCR or CD40 stimulation (Vanden Bush, T. J. and G. A. Bishop, TLR7 and CD40 cooperate in IL-6 production via enhanced INK and AP-1 activation. Eur J Immunol, 2008. 38(2): p. 400-9; Haxhinasto, S. A. and G. A. Bishop, Synergistic B cell activation by CD40 and the B cell antigen receptor: role of B lymphocyte antigen receptor-mediated kinase activation and tumor necrosis factor receptor-associated factor regulation. J Biol Chem, 2004. 279(4): p. 2575-82). More recently, TLR4-stimulating adjuvants have been approved for human use. AS04, an adjuvant containing MPL, a TLR4 ligand, plus alum is currently approved for use in Cervarix, the human papilloma virus (HPV) vaccine, and Fendrix, the hepatitis B virus (HBV) vaccine (Casella, C. R. and T. C. Mitchell, Putting endotoxin to work for us: monophosphoryl lipid A as a safe and effective vaccine adjuvant. Cell Mol Life Sci, 2008. 65(20): p. 3231-40; Didierlaurent, A. M., et al., AS04, an aluminum salt- and TLR4 agonist-based adjuvant system, induces a transient localized innate immune response leading to enhanced adaptive immunity. J Immunol, 2009. 183(10): p. 6186-97). Additionally, vaccines utilizing TLR3 and TLR9 agonists as adjuvants are currently in phase III clinical trials (Sogaard, O. S., et al., Improving the immunogenicity of pneumococcal conjugate vaccine in HIV-infected adults with a toll-like receptor 9 agonist adjuvant: a randomized, controlled trial. Clin Infect Dis, 2010. 51(1): p. 42-50; Halperin, S. A., et al., Comparison of the safety and immunogenicity of hepatitis B virus surface antigen co-administered with an immunostimulatory phosphorothioate oligonucleotide and a licensed hepatitis B vaccine in healthy young adults. Vaccine, 2006. 24(1): p. 20-6). To identify clinically relevant B cell vaccine (Bvac) stimulation strategies and to optimize the potential for B lymphocyte Ag presentation and immunotherapy, the CD8⁺ T cell stimulatory capacities of differentially stimulated Ag-pulsed B cells were compared and this investigation was extended to in vivo responses to both infectious pathogens and cancer.

Trafficking of Activated B Cells after Intravenous Injection

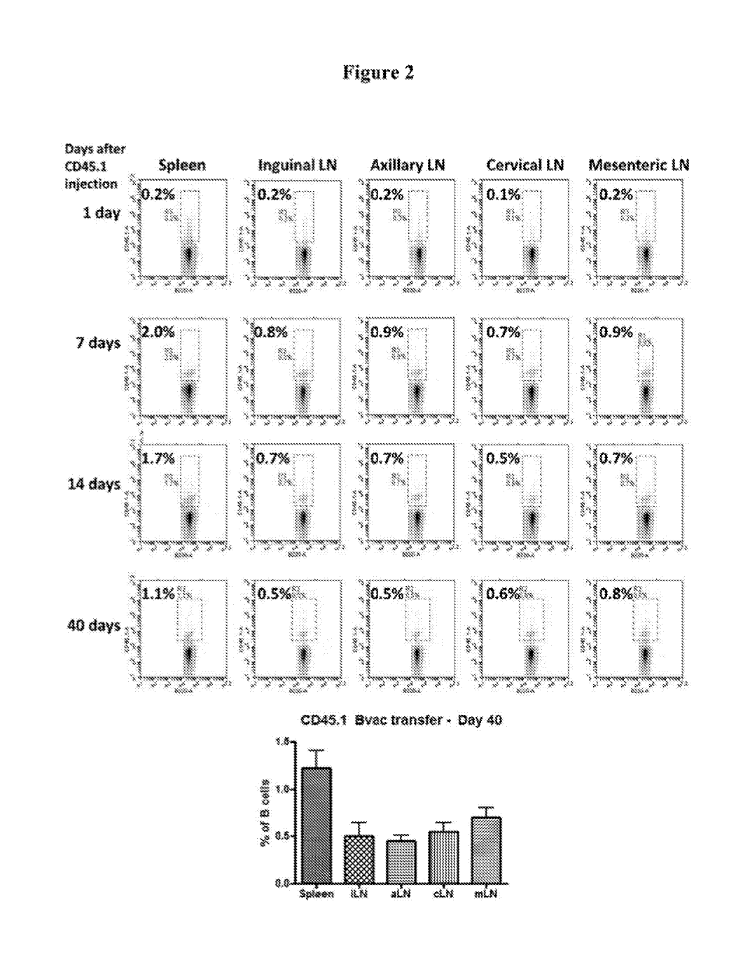

In order to optimize B lymphocytes to act as antigen-presenting cells in cellular vaccines, it is necessary to know where the injected cells go and how long they persist. CD45.1⁺ B cells were activated through CD40 and TLR4 for 24 hours in culture, and 1×10⁶ B cells were injected intravenously into naïve, congenic CD45.2⁺ WT C57BL/6 mice. 24 hours after injection, CD45.1⁺ B cells were detectable at low levels in secondary lymphoid organs, including the spleen and all lymph nodes tested (inguinal, axillary, cervical, and mesenteric) (FIG. 2). One week after injection, the CD45.1⁺ B cell population had expanded four- to ten-fold in secondary lymphoid organs, and contraction of this population was first observed at two weeks after injection (FIG. 2). At 40 days after injection (considered a CD8⁺ T cell memory time point after vaccination (Kaech, S. M., et al., Molecular and functional profiling of memory CD8 T cell differentiation. Cell, 2002. 111(6): p. 837-51) the CD45.1⁺ B cell population had contracted even further, but still maintained a sizeable population in both the spleen and lymph nodes (˜1% of all B cells in the spleen and ˜0.5% of all B cells in the lymph nodes) (FIG. 2).

Determining the Optimal Stimuli to Activate Bvacs in a Model of Murine Melanoma

While Bvacs are effective in a model of bacterial clearance, using cellular vaccines against infectious disease is unlikely to be clinically feasible as this approach is more time-consuming and less cost-effective than other commonly used vaccines and preventative treatments against bacterial and viral pathogens. Using Bvacs against cancer is an innovative therapy that can be personalized with each individual patient's cells and specific tumor antigens. B cells can be readily collected from peripheral blood in patients being treated for most kinds of cancer (although B cell lymphoma cancer treatments involving the α-CD20 mAb Rituximab may deplete peripheral B cells to the extent that this may not be a viable treatment possibility in someone currently undergoing this treatment) (Abulayha, A. M., et al., Depletion of peripheral blood B cells with Rituximab and phenotype characterization of the recovering population in a patient with follicular lymphoma. Leuk Res, 2010. 34(3): p. 307-11). The Bvac approach has particular promise for solid tumors.

To study Bvac efficacy in a model of cancer, the well-characterized murine melanoma cell line B16 was selected. This tumor grows and metastasizes similarly to human melanoma. Upon subcutaneous injection, B16 forms a palpable tumor within 10-12 days, while B16 injected intravenously mimics a model of metastasis with tumor spots appearing in the lungs and potentially liver within 18 days (Fidler, I. J., Selection of successive tumour lines for metastasis. Nat New Biol, 1973. 242(118): p. 148-9; Overwijk, W. W. and N. P. Restifo, B16 as a mouse model for human melanoma. Curr Protoc Immunol, 2001. Chapter 20: p. Unit 20.1). This cancer cell line is well-studied, with many different reagents and known antigenic peptides available for experimental use. The parent line B16F1, and a subclone that expresses chicken ovalbumin (B16-OVA) were utilized. Using B16-OVA allowed further testing of the experimental model using the immunodominant epitope SIINFEKL. Many reagents have been developed for the SIINFEKL/OVA system, including specific tetramers and the modified infectious pathogens and cancer cell lines mentioned here. Use of the parent B16F1 cells requires immunization with a peptide native to melanocytes and melanoma cells, tyrosinase. This model is more biologically relevant for vaccinating against a peptide found in both human and murine melanoma cells.

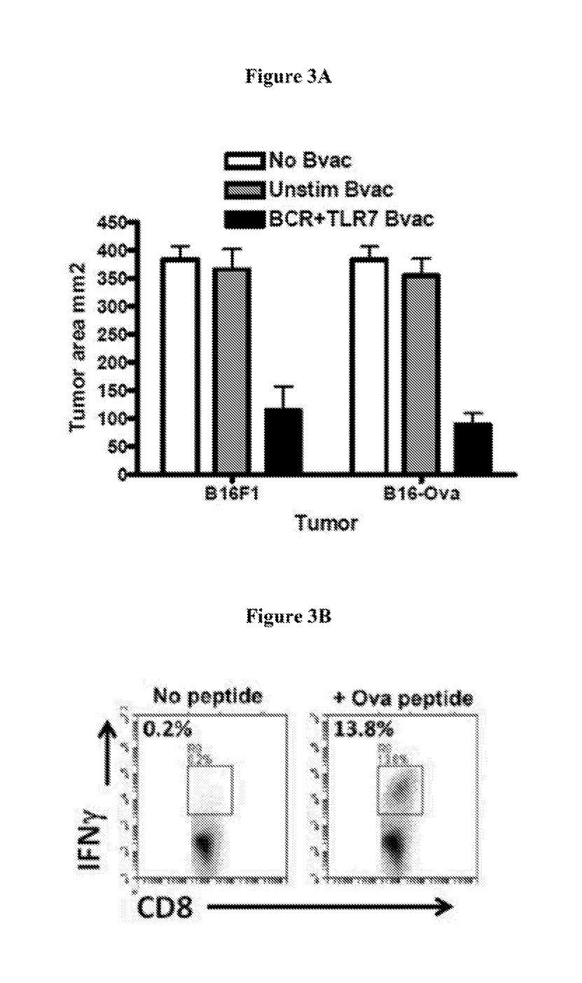

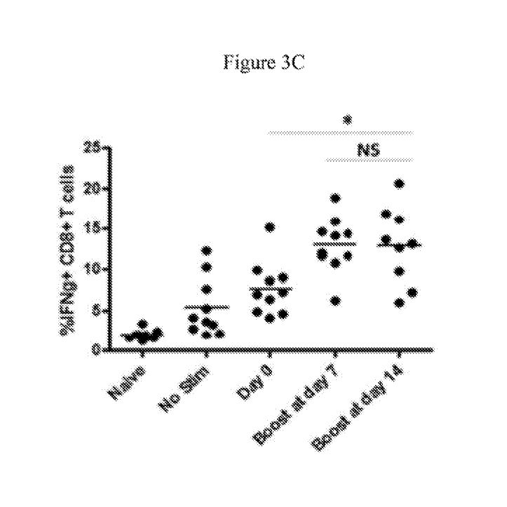

To investigate whether Bvacs would be protective in a model of murine melanoma, mice were vaccinated with Bvacs activated through the BCR and TLR7, the optimal stimulus as defined above in an infectious disease model, and pulsed with either tyrosinase or SIINFEKL peptide. Groups containing unvaccinated mice and mice vaccinated with B cells that were unstimulated yet pulsed with peptide were used as negative controls. Forty days after vaccination, mice that received tyrosinase-pulsed Bvacs were injected subcutaneously with B16F1, and mice that received SIINFEKL Bvacs were injected subcutaneously with B16-OVA. Tumor growth was measured every other day after melanoma injection. Eighteen days after tumor administration, unvaccinated mice and mice that received unactivated Bvacs reached critical tumor burden and were euthanized. Mice that received a BCR+TLR7-activated Bvac, however, displayed significantly reduced tumor burden in both tumor models (B16F1 and B16-OVA) (FIGS. 3A-C). This led to the conclusion that Bvacs could be efficacious in protection against murine melanoma in both the more relevant B16F1/tyrosinase model as well as the more artificial B16-OVA/SIINFEKL model.

The “optimal” BCR and TLR7 activation cocktail for Bvacs was defined in a model using an infectious pathogen. Immune responses against infectious pathogens differ from immune responses against cancer, and it was desired to investigate whether the BCR+TLR7 activation protocol was also optimal for creating the most effective Bvacs against cancer. Additionally, following the completion of the previously described experiments, new adjuvants were approved for human use. AS04, an adjuvant containing alum plus MPL, a TLR4 ligand, is currently approved for use in Cervarix, the human papilloma virus (HPV) vaccine, and Fendrix, the hepatitis B virus (HBV) vaccine (Casella, C. R. and T. C. Mitchell, Putting endotoxin to work for us: monophosphoryl lipid A as a safe and effective vaccine adjuvant. Cell Mol Life Sci, 2008. 65(20): p. 3231-40). Furthermore, vaccines with adjuvants utilizing TLR9 agonists are currently in late-stage clinical trials, including new formulations of hepatitis B and pneumococcal vaccines (Sogaard, O. S., et al., Improving the immunogenicity of pneumococcal conjugate vaccine in HIV-infected adults with a toll-like receptor 9 agonist adjuvant: a randomized, controlled trial. Clin Infect Dis, 2010. 51(1): p. 42-50.; Halperin, S. A., et al., Comparison of the safety and immunogenicity of hepatitis B virus surface antigen co-administered with an immunostimulatory phosphorothioate oligonucleotide and a licensed hepatitis B vaccine in healthy young adults. Vaccine, 2006. 24(1): p. 20-6). As stimulation through TLR4 or TLR9 highly activates murine B cells, we also included Bvac groups activated through these TLRs in addition to activation strategies that we had tested in Bvacs against Listeria.

To test these different Bvac activating stimuli, an approach very similar to that in FIGS. 3A-3C was utilized. B cells were activated in culture with the indicated stimuli, pulsed with SIINFEKL peptide, and injected into mice. A prime-boost system was used in this experiment to boost the immune response from our Bvac. Mice were given a second, identical Bvac 14 days after the first vaccine. Forty days after the boost, mice were injected subcutaneously with a low dose of B16-OVA. Tumors were measured every other day for 60 days. Naïve, unvaccinated mice developed palpable tumors within ten to twenty days after tumor injection and reached critical tumor burden within 16 to 24 days. Tumor development and progression was delayed in all Bvac treated mice, regardless of the activating stimulation (FIG. 4). However, two stimulations, BCR+TLR7 and CD40+TLR4, were significantly superior to the others tested in preventing tumor growth and promoting mouse survival. These stimulations did not significantly differ from each other, but responses to CD40+TLR4 activated Bvacs approached significance over those to BCR+TLR7 activated Bvacs. From these data, it was concluded that Bvacs can be optimized to effectively prevent and/or delay tumor development.

### CONCLUSIONS

Intravenously injected activated B cells traffic to secondary lymph nodes, expand, and persist for at least 40 days after injection, maintaining a sizeable compartment of CD45.1⁺ cells weeks after the initial injection of activated B lymphocytes (FIG. 2). No autoimmune symptoms have been observed in mice receiving Bvacs.

BCR+TLR7-stimulated Bvacs were efficient in delaying tumor growth in two models of B16 melanoma: parent B16F1 cells, and B16 cells that express chicken ovalbumin (FIGS. 3A-3C). In addition to BCR+TLR7-stimulated Bvacs, an additional Bvac stimulus was identified that provides effective protection against tumor development and growth: stimulation through CD40 and TLR4. There was no significant difference in the ability of CD40+TLR4-activated Bvacs and BCR+TLR7-activated Bvacs in preventing and delaying tumor growth after injection of B16 melanoma (FIG. 4). However, CD40+TLR4-activated Bvacs showed better responses in early time points after tumor injection, and repeating this experiment may make these differences statistically significant. CD40+TLR4 stimulation of B cells has been previously validated in studies using B cells from tumor-draining lymph nodes. These B cells were collected, stimulated through CD40 and TLR4, and injected back into tumor bearing hosts whereupon the B cells induced tumor-specific T cell immunity and tumor regression.

From these data, it is concluded that B cells can be activated to act as effective APCs in cellular vaccines, providing protection against both infectious pathogens and cancer.

### Example 2

The data in Example 1 were obtained with a line of the mouse melanoma called B16-OVA, which expressed the foreign antigen ovalbumin. The advantage of this tumor is that mice are available that are transgenic for a T cell antigen receptor (TCR) that recognizes an OVA peptide. That is, every T cell in the mouse recognizes the same antigenic peptide, expressed by the tumor. This facilitates analyzing the T cell response to the tumor. However, it is a very artificial situation, compared to a tumor that a patient will present in the clinic.

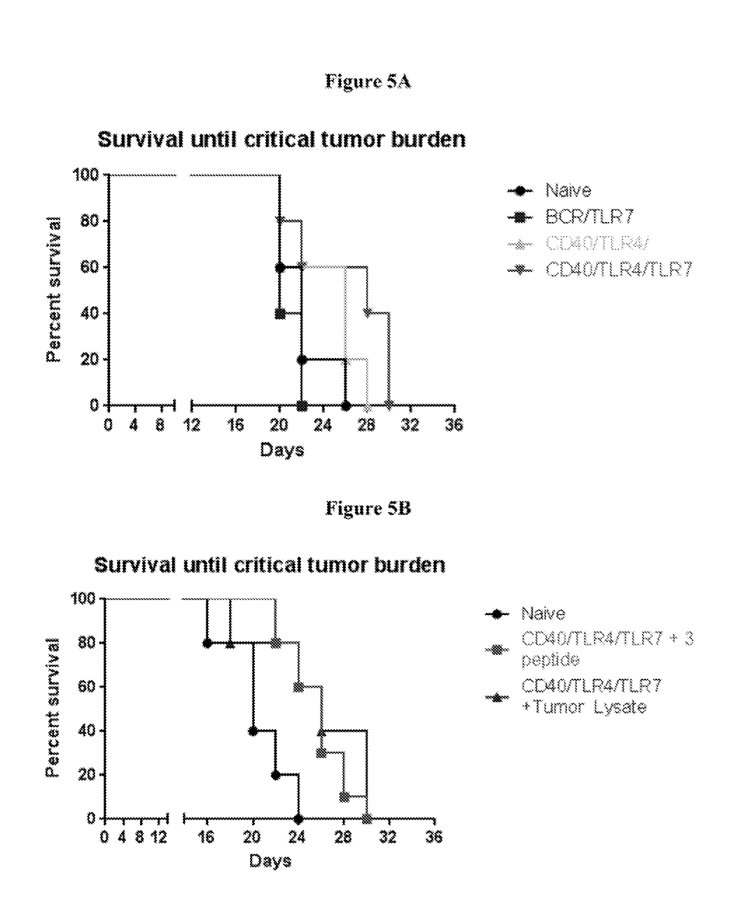

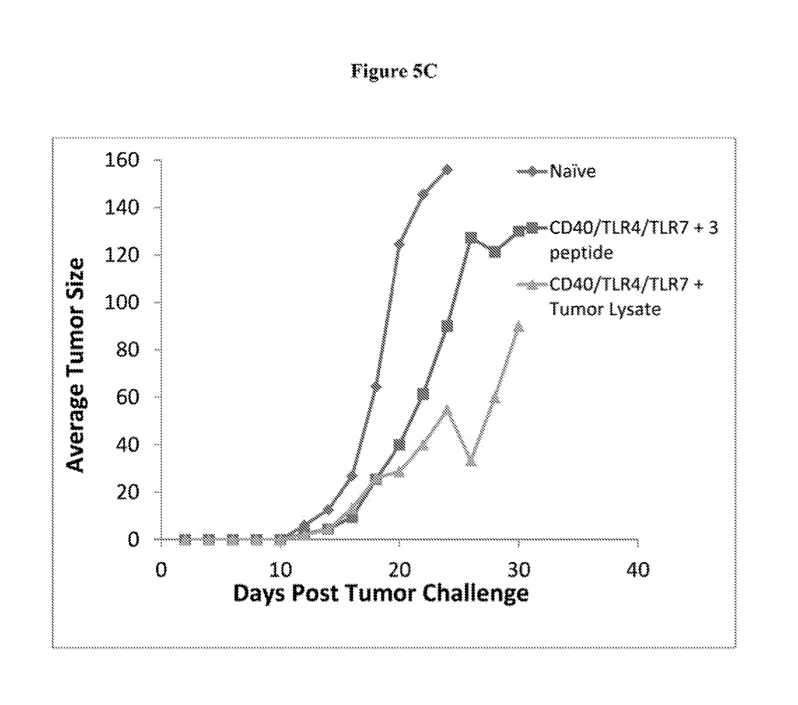

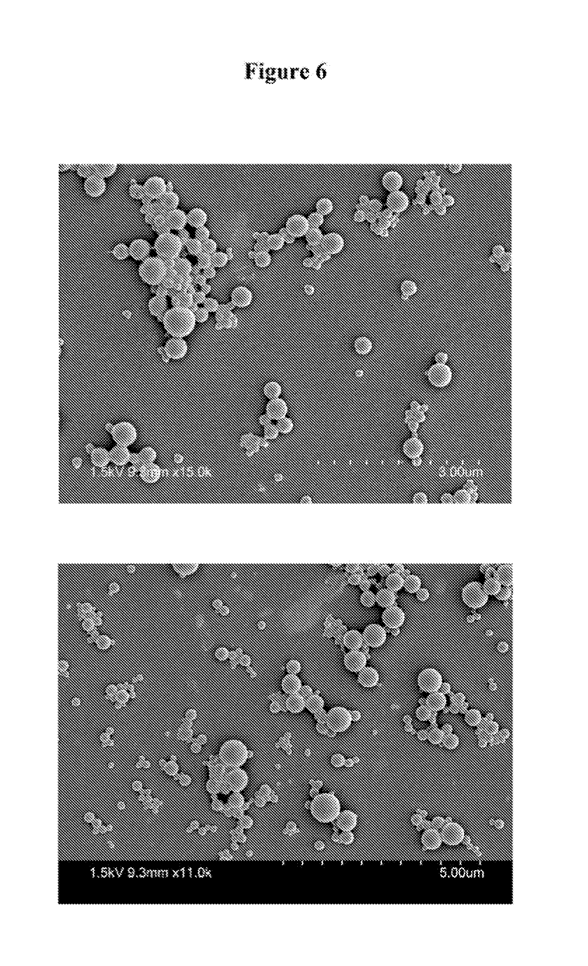

Further studies were performed using the B16F1 tumor, in order to move the Bvac approach closer to translation by mimicking a situation more similar to the clinical reality in human melanoma, which does not express a strongly immunogenic foreign Ag like OVA. A known purified melanoma-derived peptide, TRP-2, was delivered with different combinations of TLR and BCR/CD40 stimuli. The immunizing antigen was a mixture of melanoma peptides (TRP-2, Mage-A5 and Mage-Ax) (FIG. 5B). Although all the mice ultimately died (B16F1 is a highly aggressive tumor, similar to human melanoma), the chosen “optimal” adjuvant combination (ligands for CD40, TLR4 and TLR7) gave the most prolonged survival.

In a further experiment, the focus was on determining the optimal adjuvant combination only. Two antigen sources were compared: 3 purified melanoma peptides or a simple tumor cell lysate, prepared according to established protocols. Surprisingly, the results showed that the lysate (an approach that could easily be used for ANY patient tumor, because the amount of lysate needed is easily obtained in a tumor resection or even just a biopsy) was as effective an immunogen as the peptides (FIG. 5A). In fact, the line graph shows that tumor size was actually significantly SMALLER with the tumor lysate (FIG. 5C).

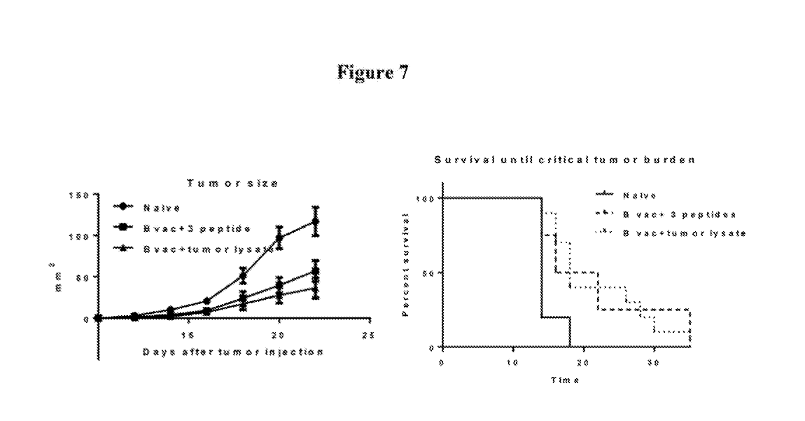

The most effective stimulus combination was delivered through a combination of peptides plus signals through CD40, TLR4 and TLR7. Concentrating on this effective combination, it was tested whether a mixture of purified melanoma peptides (TRP-2, Mage-A5 and Mage-Ax) would be more effective than a single antigenic peptide in producing a protective Bvac. Most important, tumor-derived cellular lysate preparations were tested as a source of tumor antigen. This could obviate a need for known tumor antigens, as the modest amount of cell lysate required would be easily obtained from a biopsy sample, or tissue obtained during a tumor resection. Encouragingly, the tumor lysate actually provided better results than a combination of three purified endogenous melanoma-specific peptides (FIG. 7), resulting in both smaller tumors, enhanced time to tumor development (FIG. 7), and delayed tumor onset (not shown). Thus, using tumor lysate as a source of Ag is preferred. There were several long-term tumor-free survivors in the group receiving tumor-lysate-stimulated Bvac.

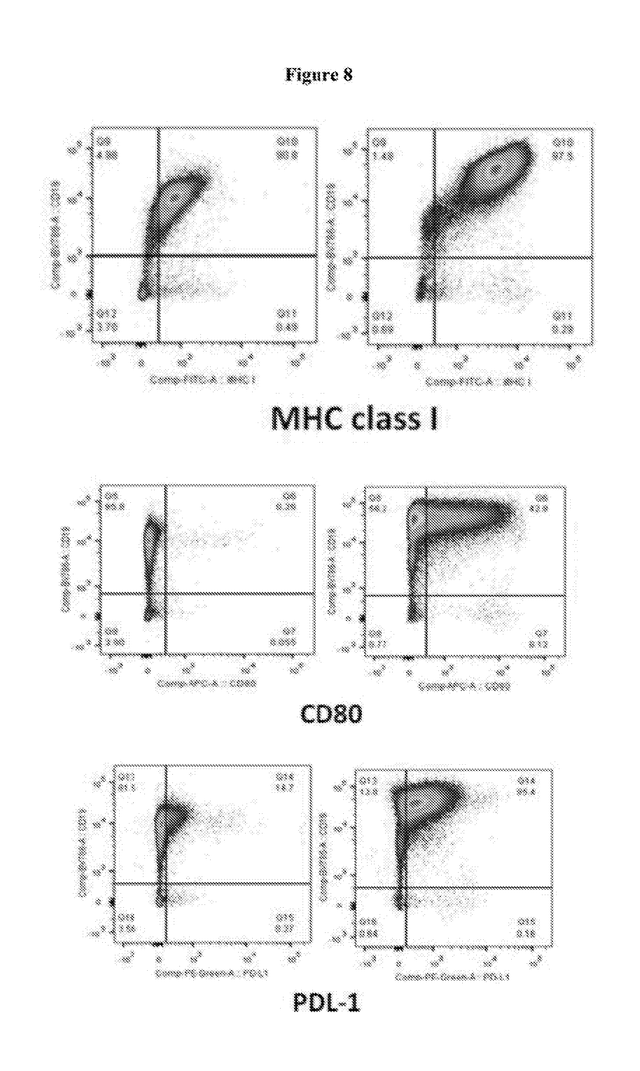

Surface molecule upregulation and cytokine production as potential biomarkers of effective Bvac stimulation was also investigated. For surface molecule upregulation, a strong correlation between the most effective Bvac stimulus (CD40+TLR4+TLR7+tumor lysate) and the best upregulation of CD80, MHC class I and II, and adhesion molecules was observed. Interestingly, this stimulus also induced the highest expression of the checkpoint inhibitor PD-L1 by the B cells (FIG. 8).

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of preparing an anti-cancer composition comprising activated immune cells and a pharmaceutically acceptable excipient, the method comprising contacting immune cells obtained from an animal with an activating agent and with an immunogenic material obtained from the animal to form the activated cells, wherein the activated immune cells are a population of cells that are at least 90% B-lymphocytes.

2. A method of preparing an anti-cancer composition comprising activated cells and a pharmaceutically acceptable excipient, the method comprising:
obtaining immune cells from an animal,
obtaining immunogenic material from the animal,
contacting the cells with an activating agent, and
contacting the cells with the immunogenic material to generate the activated cells,
wherein the activated immune cells are a population of cells that are at least 90% B-lymphocytes.

3. The method of claim 1 or claim 2, further comprising purifying the activated cells away from the activating agent and the immunogenic material.

4. The method of any one of claims 1-3, wherein the immune cells are a population of cells that are at least 95% B-lymphocytes.

5. The method of any one of claims 1-3, wherein the immune cells are a population of cells that are at least 99% B-lymphocytes.

6. The method of any one of claims 1-3, wherein the immune cells are a population of cells that are 100% B-lymphocytes.

7. The method of any one of claims 1-6, wherein the immune cells have been expanded in vitro prior to administration into an animal.

8. The method of any one of claims 1-7, wherein the immune cells are stimulated for about 1-72 hours with the activating agent and/or the immunogenic material.

9. The method of claim 8, wherein the immune cells are stimulated for about 12-60 hours with the activating agent and/or the immunogenic material.

10. The method of claim 8, wherein the immune cells are stimulated for about 24-48 hours with the activating agent and/or the immunogenic material.

11. The method of any one of claims 1-10, wherein the immune cells are obtained from peripheral blood from the animal.

12. The method of any one of claims 1-11, wherein the activating agent comprises a CD40 agonist, BCR agonist, toll-like receptor 7 (TLR7) agonist, a TLR4 agonist, AS04, alum, TLR3 agonist, and/or a TLR9 agonist.

13. The method of any one of claims 1-11, wherein the activating agent comprises a CD40 agonist, TLR7 agonist, and a TLR4 agonist.

14. The method of any one of claims 1-13, wherein the immunogenic material comprises tumor cells or a tumor cell lysate from the animal.

15. The method of claim 14, wherein the tumor cells are solid tumor cells or are hematopoietic cancer cells.

16. The method of claim 14 or 15, wherein the tumor cells are lysed.

17. The method of any one of claims 14-16, wherein the tumor cells are irradiated.

18. The method of any one of claims 1-17, wherein the activating agent is encapsulated in a nanoparticle.

19. The method of any one of claims 1-18, wherein the immunogenic material is encapsulated in a nanoparticle.

20. The method of claim 18 or 19, wherein the nanoparticle encapsulating the activating agent and/or the nanoparticle encapsulating the immunogenic material comprises Poly(Lactide-co-Glycolide) (PLGA).

21. The method of any one of claims 1-20, wherein the anti-cancer composition further comprises an anti-cancer therapeutic.

22. A pharmaceutical composition comprising a pharmaceutically acceptable excipient and immune cells obtained from an animal, wherein the cells have been stimulated ex vivo with an activating agent and have been stimulated ex vivo with an immunogenic material obtained from the animal for the therapeutic treatment of cancer, wherein the activated immune cells are a population of cells that are at least 90% B-lymphocytes.

23. The composition of claim 22 substantially devoid of activating agent and/or immunogenic material.

24. The composition of claim 22 or 23, wherein the immune cells are a population of cells that are at least 95% B-lymphocytes.

25. The composition of claim 22 or 23, wherein the immune cells are a population of cells that are at least 99% B-lymphocytes.

26. The composition of claim 22 or 23, wherein the immune cells are a population of cells that are 100% B-lymphocytes.

27. The composition of any one of claims 22-26, wherein the immune cells have been expanded in vitro prior to administration into an animal.

28. The composition of any one of claims 22-27, wherein the immune cells are stimulated for about 1-72 hours with the activating agent and/or the immunogenic material.

29. The composition of any one of claims 22-27, wherein the immune cells are stimulated for about 12-60 hours with the activating agent and/or the immunogenic material.

30. The composition of any one of claims 22-27, wherein the immune cells are stimulated for about 24-48 hours with the activating agent and/or the immunogenic material.

31. The composition of any one of claims 22-30, wherein the immune cells are obtained from peripheral blood from the animal.

32. The composition of any one of claims 22-31, wherein the activating agent comprises a CD40 agonist, BCR agonist, toll-like receptor 7 (TLR7) agonist, a TLR4 agonist, AS04, alum, TLR3 agonist, and/or a TLR9 agonist.

33. The composition of any one of claims 22-31, wherein the activating agent comprises a CD40 agonist, TLR7 agonist, and a TLR4 agonist.

34. The composition of any one of claims 22-33, wherein the immunogenic material comprises tumor cells or a tumor cell lysate from the animal.

35. The composition of claim 34, wherein the tumor cells are solid tumor cells or are hematopoietic cancer cells.

36. The composition of claim 34 or 35, wherein the tumor cells are lysed.

37. The composition of any one of claims 34-36, wherein the tumor cells are irradiated.

38. The composition of any one of claims 22-37, wherein the activating agent is encapsulated in a nanoparticle.

39. The composition of any one of claims 22-37, wherein the immunogenic material is encapsulated in a nanoparticle.

40. The composition of claim 38 or 39, wherein the nanoparticle encapsulating the activating agent and/or the nanoparticle encapsulating the immunogenic material comprises Poly(Lactide-co-Glycolide) (PLGA).

41. The composition of any one of claims 22-40, wherein the pharmaceutical composition further comprises an anti-cancer therapeutic.

42. The composition of any one of claims 22-41, further comprising a physiologically-acceptable, non-toxic vehicle.

43. The composition of any one of claims 22-42, wherein the pharmaceutical composition further comprises an adjuvant.

44. The composition of claim 43, wherein the adjuvant is a TLR ligand.

45. The composition of claim 43, wherein the adjuvant is a non-TLR ligand that stimulates an immune response.

46. A method of treating cancer in an animal in need thereof comprising, administering to the animal a cancer vaccine and the pharmaceutical composition of any one of claims 22-45.

47. A method comprising administering a dendritic cell-based vaccine followed by administering the pharmaceutical composition of any one of claims 22-45 to an animal in need thereof.

48. The method of claim 46 or 47, further comprising administering repeat dosages of the pharmaceutical composition of any one of claims 22-45.

49. The method of any one of claims 46-48, wherein the administration is by means of injection.

50. A method of eliciting an immune response in an animal in need thereof, comprising administering to the animal the pharmaceutical composition of any one of claims 22-45.

51. The method of claim 50, wherein the composition is administered intravenously or intra-tumorally.

52. The method of claim 50 or 51, wherein the pharmaceutical composition is administered at more than one time point.

53. The method of claim 52, wherein the pharmaceutical composition is administered multiple times.

54. The method of claim 53, wherein the pharmaceutical composition is administered two to five times.

55. The method of any one of claims 46-54, wherein the animal is a mammal.

56. The method of claim 55, wherein the mammal is a human.

57. A use of the pharmaceutical composition of any one of claims 22-45 for treating cancer.

58. A use of the pharmaceutical composition of any one of claims 22-45 to prepare a medicament for treating cancer in an animal.
